(19) United States
(12) Plant Patent Application Publication
(10) Pub. No.: US 2010/0186133 P1
(43) Pub. Date: Jul. 22, 2010

(54) Phalaenopsis Plant Named 'The Pride Of Taiwan'

(50) ;

(76) Inventor: Tin-Fan Ho, Taipei City (TW)

Correspondence Address: FRENKEL & ASSOCIATES, 3975 UNIVERSITY DR., STE. 330, FAIRFAX, VA, 22030, US

(21) Appl. No.: 12/321,177

(22) Filed: Jan. 19, 2009

(51) Int. Cl. A01H 5/00

(52) U.S. Cl. PLT/311

(57) ABSTRACT

A new and distinct cultivar of Phalaenopsis plant named ‘The Pride of Taiwan’, particularly characterized by its attractive and unique light green flowers with light pink shaded petals, freely flowering habit, freely branching and thick flowering stems, and strong growth ability.

### FIELD OF THE INVENTION

### Botanical Classification: Phalaenopsis sp.

### Variety Denomination: ‘The Pride of Taiwan’.

The present invention relates to botanical classification/cultivar designation: Phalaenopsis Orchid cultivar The Pride of Taiwan.

### BACKGROUND OF THE INVENTION

The present invention comprises a new and distinct cultivar of Phalaenopsis Orchid, and hereinafter referred to by the cultivar name, The Pride of Taiwan.

The new cultivar is a product of a planned breeding program conducted by the inventor in Hualien, Taiwan. The objective of the breeding program is to create new uniform pot-type Phalaenopsis Orchid cultivars having attractive flower coloration.

The new cultivar breeder was Tin-Fan Ho. The new cultivar was discovered by the inventor from within the progeny of a cross-pollination of two identified proprietary selections of Phalaenopsis Orchid, not patented, on April, 1998, in a controlled environment in Taiwan. Later, it was verified and registered in Royal Horticulture Society (R.H.S.) and had its variety name “P. Ho's Colourful Bubbles” on September, 2001.

Asexual propagation by tissue culture in a laboratory in Hualien, Taiwan has been used to increase the number of plants for evaluation and has demonstrated that the unique combination of characteristics as herein disclosed for the new Phalaenopsis Orchid are firmly fixed and are retained through successive generations of asexual reproduction.

### SUMMARY OF THE INVENTION

The following traits have been repeatedly observed and are determined to be basic characteristics of ‘The Pride of Taiwan’ which in combination distinguish this Phalaenopsis Orchid as a new and distinct cultivar:

1. Light green flowers with light pink shaded petals.

2. ‘The Pride of Taiwan’ has much more branching and flowers than other green phalaenopsis.

3. Strong growth ability and disease resistance.

4. Compound raceme type of inflorescence and thick flowering stem.

Plant of the new cultivar differs primarily from plants of the parent cultivars in the flower color.

Plant of new Phalaenopsis Orchid can be compared to plants of the cultivar P. Fortune Saltzman ‘Maple Bridge’ (not patented). Plants of the new Phalaenopsis Orchid differed from plants of the cultivar P. Fortune Saltzman ‘Maple Bridge’ in the following characteristics:

1. Plant of the new Phalaenopsis was bigger than plants of the cultivar P. Fortune Saltzman ‘Maple Bridge’.

2. Plant of the new Phalaenopsis has much more branching and flowers than plants of the cultivar P. Fortune Saltzman ‘Maple Bridge’.

3. Plant of the new Phalaenopsis has much more roots than plants of the cultivar P. Fortune Saltzman ‘Maple Bridge’.

4. Plant of the new Phalaenopsis has stronger disease resistance than plants of the cultivar P. Fortune Saltzman ‘Maple Bridge’.

### BRIEF DESCRIPTION OF THE DRAWINGS

Colors in the photographs may appear different from the color values that appear in the detailed botanical description which more accurately describe the new cultivar.

FIG. 1 is a side view of a plant of ‘The Pride of Taiwan’ flowering in the pot of 13 cm.

FIG. 2 is a close-up view showing the characteristics of the flower.

FIG. 3 is a close-up view showing the characteristics of the leaf.

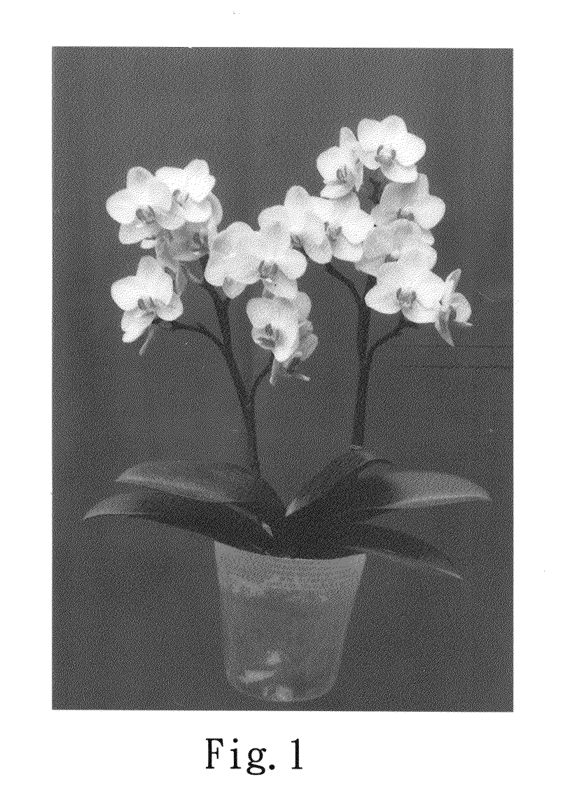

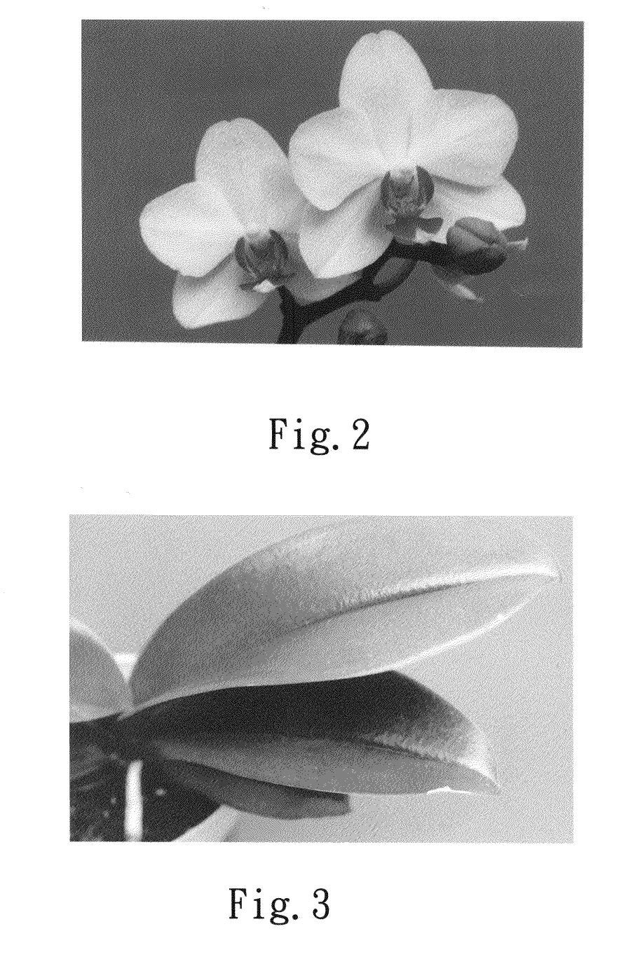

### DETAILED DESCRIPTION OF THE PREFERRED EMBODIMENTS

‘The Pride of Taiwan’ has not been observed under all possible environmental conditions. The phenotype may vary significantly with variations in environment such as temperature and light intensity, without however, any change in genotype.

In the following description, color references are made to The Royal Horticultural Society (R.H.S.) Colour Chart.

Plants used for the aforementioned photographs and following detailed botanical description were 12 months in maturity and grown in the pot of 13 cm, in Hualien, Taiwan, in a controlled greenhouse with day temperatures about 18 to 32° C., night temperatures about 18 to 32° C., and light levels about 11,000 to 16,000 lux. The photographs and the detailed botanical description were taken from February to April.
Parentage:
   Seed.—P. Ho's Little Caroline, not patented.
   Pollen.—P. Dou-dii Golden Princess, not patented.
Propagation: Asexual propagation by tissue culture.
Plant description:
   Plant shape.—Two-ranked leaves affixed to a short central stem (monopodial growth). Single flowers arranged on upright and sturdy flowering compound racemes.
   Plant height, soil level to top of foliar plane.—About 10 to 15 cm.
   Plant height, soil level to top of inflorescences.—About 35 to 40 cm.
   Plant diameter.—About 25 to 30 cm.
Flowers per stem: Approximately 20 to 60.
Foliage description:
   Quantity per plant.—About 8 to 9.
   Length.—About 15 to 18 cm.
   Width.—About 5.8 to 7.3 cm.
   Shape.—Oblong.
   Apex.—Oblong.
   Base.—Oblong.
   Margin.—Oblong.
   Aspect.—Oblong.
   Texture, upper and lower surfaces.—Thick.
   Venation.—One clear vein.
   Color.—Upper surface — RHS 131A.
   Color.—Lower surface — RHS 135C.
Flower description:
   Flower type.—Single zygomorphic flowers, roughly pentagonal in shape.
   Flower arrangement.—Compound racemes.
   Flowering stems.—Upright, freely branching and thick.
   Flowering habit.—Plants freely flowering; plants typically produce four to eight branched flowering stems with at least 20 to 30 flowers each.
   Fragrance.—Flowers not fragrant.
   Natural flowering season.—From February to June in Hualien, Taiwan.
   Post-production longevity.—Plants of the ‘The Pride of Taiwan’ maintain good leaf and flower substance for about two to four months on the plant under interior environmental conditions.
   Inflorescence length.—About 30 to 40 cm.
   Inflorescence diameter.—About 20 to 30 cm.
Flower bud:
   Flower bud shape.—Orbicular.
   Flower bud length.—About 2 cm.
   Flower bud diameter.—About 1.8 cm.
   Flower bud color.—About RHS 154D.
   Flower diameter.—About 6 to 6.5 cm.
   Flower depth.—About 2.5 cm.
Petals:
   Number of petals.—Three per flower.
   Length.—About 2.8 cm.
   Diameter.—About 2.8 cm.
   Shape.—Semicircular.
   Apex.—Elliptical.
   Base.—Rhombate.
   Margin.—Semicircular.
   Texture, upper and lower surfaces.—Smooth.
   Petal main color.—RHS 154D.
   Petal pattern color.—RHS 62B.
Labellum:
   Length.—About 2 cm.
   Diameter.—About 1.2 cm.
   Shape.—Rhombate with no absent.
   Base color of the apical lobe.—RHS 59B.
   Tip color of the apical lobe.—RHS N74B.
Sepals:
   Quantity.—Three per flower.
   Dorsal sepal length.—About 3.1 cm.
   Lateral sepal length.—About 3.1 cm.
   Dorsal sepal diameter.—About 2.7 cm.
   Lateral sepal diameter.—About 2.6 cm.
   Shape.—Elliptical.
   Apex.—Elliptical.
   Base.—Elliptical.
   Margin.—Elliptical.
   Texture, upper and lower surfaces.—Smooth.
   Dorsal sepal main color.—RHS 154D.
   Dorsal sepal pattern color.—RHS 154D.
   Lateral sepal main color.—RHS 154D.
   Lateral sepal pattern color.—RHS 154D.
Peduncles:
   Length.—About 35 to 40 cm.
   Diameter.—About 6 mm.
   Aspect.—Thick.
   Strength.—Strong.
   Texture.—Thick.
   Color.—RHS 131A.
Pedicels:
   Length.—About 15 cm.
   Diameter.—About 4 mm.
   Aspect.—About 55° from vertical.
   Strength.—Thick.
   Texture.—Strong.
   Color.—RHS 131A.
   Color towards the base.—RHS 131A.
Reproductive organs: The stamens, style and stigmas are fused into a single, short structure called the column, possessing one terminal anther with pollen grains united into a pollinia, which are covered by an anther cap. The stigma is located under the column behind the pollinia. The ovary is inferior with three carpels presented. The plant has not produced seed.
Column:
   Length.—About 0.9 cm.
   Diameter.—About 4 mm.
   Color.—RHS 3B.
   Color towards to apex.—RHS 3B.
Pollinia:
   Quantity of pollen masses.—Two pollen masses.
   Diameter.—About 1 mm.
   Color.—RHS 23B.
Ovary:
   Length.—About 30 mm.
   Diameter.—About 2.5 mm.
   Color.—RHS 131A.
Root: It takes 30 days for plants growing in tissue culture to initiate roots.
   Diameter.—About 6 mm.
   Color.—RHS 190D.
Disease/pest resistance: Strong disease resistance.
Temperature tolerance: Plants of the new Phalaenopsis Orchid have been observed to be tolerant to temperatures from about 15 to 35° C.

What is claimed is:

1. A new and distinct cultivar of Phalaenopsis orchid plant named ‘The Pride of Taiwan’ as illustrated and described.
